(19) United States
(12) Patent Application Publication
(10) Pub. No.: US 2023/0330296 A1
(43) Pub. Date: Oct. 19, 2023

(54) SPACE CLEANING DEVICE AND SPACE CLEANING SYSTEM USING SAME

(71) Applicant: Panasonic Intellectual Property Management Co., Ltd., Osaka (JP)

(72) Inventors: Ryousuke SUGA, Aichi (JP); Tomohiro HAYASHI, Aichi (JP); Yuki MIZUNO, Aichi (JP); Yoko ISHIDA, Aichi (JP); Shinji YOSHIDA, Aichi (JP)

(21) Appl. No.: 18/245,673

(22) PCT Filed: Sep. 30, 2021
(86) PCT No.: PCT/JP2021/036122

§ 371 Date: Mar. 16, 2023

(30) Foreign Application Priority Data
Oct. 30, 2020 (JP) 2020-182017

(51) Int. Cl. A61L 9/14; C02F 1/461; C02F 1/66; F24F 3/167

(52) U.S. Cl. CPC A61L 9/14; C02F 1/4618; C02F 1/66; F24F 3/167; C02F 2001/46185; C02F 2201/4615; C02F 2201/4618; A61L 2101/06

(57) ABSTRACT

Humidifying and cleaning device of the present disclosure includes: centrifugal crushing unit that generates hypochlorous acid water micronized by a micronization operation for micronizing hypochlorous acid water stored in humidifier tank, and causes air flowing inside of the humidifying and cleaning unit to contain and release the hypochlorous acid water micronized by the micronization operation; and humidification controller that controls the micronization operation. Humidification controller is configured to perform a first treatment of draining the hypochlorous acid water stored in humidifier tank and supplying new hypochlorous acid water based on time information specified in advance during the micronization operation, that is, time information from when the micronization operation is started until a content of the hypochlorous acid contained in the hypochlorous acid water stored in humidifier tank becomes less than or equal to a reference content.

### TECHNICAL FIELD

The present disclosure relates to a space cleaning device that micronizes water, blows out sucked air containing the micronized water, and releases the micronized water containing hypochlorous acid, and a space cleaning system using the space cleaning device.

### BACKGROUND ART

As a conventional space cleaning device, an air conditioning system that sterilizes a space by bringing air to be supplied indoors into contact with a gas-liquid contact member containing the hypochlorous acid and discharging the air is known (see, for example, PTL 1).

In the conventional space cleaning device, generally, water stored in the device (water containing the hypochlorous acid) and the hypochlorous acid are vaporized and consumed along with a micronization operation. Then, when the stored water runs out, new water (water containing the hypochlorous acid) is supplied to the space cleaning device. In the conventional space cleaning device, such an operation is automatically repeated.

### CITATION LIST

### Patent Literature

PTL 1: Unexamined Japanese Patent Publication No. 2009-133521

### SUMMARY OF THE INVENTION

However, the hypochlorous acid has a higher vapor pressure than water and is easily vaporized. Therefore, the conventional space cleaning device has a problem that the hypochlorous acid contained in the stored water is vaporized and reduced before the stored water (water containing the hypochlorous acid) is consumed along with the micronization operation particularly in summer seasons when a relative humidity is high, and the hypochlorous acid is not released at a set concentration.

An object of the present disclosure is to provide a space cleaning device and a space cleaning system using the same capable of stably imparting the hypochlorous acid, in a case where the micronization operation of including the hypochlorous acid in the micronized water and releasing the hypochlorous acid is continuously performed.

A space cleaning device according to the present disclosure includes: a humidifying and cleaning unit that generates hypochlorous acid water micronized in the micronization operation of micronizing the hypochlorous acid water stored in a water storage unit, and causes air flowing inside of the humidifying and cleaning unit to contain and release the hypochlorous acid water micronized by the micronization operation; and a controller that controls the micronization operation. The controller is configured to perform a first treatment of draining the hypochlorous acid water stored in the water storage unit and supplying new hypochlorous acid water based on time information specified in advance during the micronization operation, that is, time information from when the micronization operation is started until content of the hypochlorous acid contained in the hypochlorous acid water stored in the water storage unit becomes less than or equal to a reference content.

Further, the space cleaning system according to the present disclosure includes the space cleaning device described above and a hypochlorous acid water generation device that generates the hypochlorous acid water by electrolyzing a chloride aqueous solution. The hypochlorous acid water generation device supplies the hypochlorous acid water to the water storage unit in the first treatment.

Further, the space cleaning device according to the present disclosure includes: the humidifying and cleaning unit that generates the hypochlorous acid water micronized by the micronization operation of centrifugally crushing and micronizing the hypochlorous acid water pumped from the water storage unit by a rotation of a pumping pipe, and includes and releases the hypochlorous acid water micronized by the micronization operation in the air flowing inside; and the controller that controls the micronization operation. The controller is configured to perform a wastewater treatment of the hypochlorous acid water stored in the water storage unit during the micronization operation based on the time information specified in advance, that is, the time information until the content of the hypochlorous acid contained in the hypochlorous acid water stored in the water storage unit becomes less than or equal to a reference content.

According to the present disclosure, it is possible to provide a space cleaning device and a space cleaning system using the same capable of stably imparting the hypochlorous acid in a case where the micronization operation of including the hypochlorous acid in the micronized water and releasing the hypochlorous acid is continuously performed.

### BRIEF DESCRIPTION OF THE DRAWINGS

FIG. 1 is a schematic diagram of a space cleaning system according to a first exemplary embodiment of the present disclosure.

FIG. 2 is a block diagram showing a configuration of a hypochlorous acid controller of a hypochlorous acid water generation device in the space cleaning system according to the first exemplary embodiment.

FIG. 3 is a block diagram illustrating a configuration of a humidification controller of a humidifying and cleaning device in the space cleaning system according to the first exemplary embodiment.

FIG. 4 is a schematic view showing a temporal change in a hypochlorous acid concentration in the space cleaning system according to the first exemplary embodiment.

FIG. 5 is a schematic diagram of a space cleaning system according to a second exemplary embodiment of the present disclosure.

FIG. 6 is a schematic view showing a temporal change in a hypochlorous acid concentration in the space cleaning system according to the second exemplary embodiment.

FIG. 7 is a schematic diagram of a space cleaning system according to a third exemplary embodiment of the present disclosure.

FIG. 8 is a schematic view showing a temporal change in a hypochlorous acid concentration in the space cleaning system according to the third exemplary embodiment.

### DESCRIPTION OF EMBODIMENT

A space cleaning device according to the present disclosure includes: a humidifying and cleaning unit that generates hypochlorous acid water micronized in the micronization operation of micronizing the hypochlorous acid water stored in a water storage unit, and causes air flowing inside to contain and release the micronized hypochlorous acid water; and a controller that controls the micronization operation. The controller is configured to perform a first treatment of draining the hypochlorous acid water stored in the water storage unit and supplying new hypochlorous acid water based on time information specified in advance during the micronization operation, that is, time information from when the micronization operation is started until content of the hypochlorous acid contained in the hypochlorous acid water stored in the water storage unit becomes less than or equal to a reference content.

According to such a configuration, the hypochlorous acid contained in the hypochlorous acid water stored in the water storage unit is vaporized and reduced based on the time information specified in advance, and is replaced with new hypochlorous acid water by the first treatment before the hypochlorous acid is no longer released at a set concentration from the humidifying and cleaning unit. In other words, based on the time information, the content of the hypochlorous acid contained in the hypochlorous acid water stored in the water storage unit is maintained higher than the reference content. Therefore, the hypochlorous acid of the set concentration can be stably imparted to the air released from the humidifying and cleaning unit.

Further, in the space cleaning device according to the present disclosure, the humidifying and cleaning unit generates the hypochlorous acid water micronized by the micronization operation of centrifugally crushing and micronizing the hypochlorous acid water pumped from the water storage unit by a rotation of a pumping pipe.

Thus, the hypochlorous acid water can be efficiently micronized.

Further, in the space cleaning device according to the present disclosure, the time information is specified in advance for each concentration of the hypochlorous acid water stored in the water storage unit.

Thus, the time information is set such that the content of the hypochlorous acid contained in the hypochlorous acid water stored in the water storage unit is maintained higher than the reference content. Therefore, the hypochlorous acid of the set concentration can be stably imparted to the air released from the humidifying and cleaning unit.

Further, in the space cleaning device according to the present disclosure, a pH adjusting agent for adjusting a pH of the hypochlorous acid water is added to the hypochlorous acid water stored in the water storage unit.

Thus, the hypochlorous acid can be stably imparted from the space cleaning device using the hypochlorous acid water that is easily vaporized by adjusting the pH of the hypochlorous acid water.

Further, the space cleaning system according to the present disclosure includes the space cleaning device described above and a hypochlorous acid water generation device that generates the hypochlorous acid water by electrolyzing a chloride aqueous solution. The hypochlorous acid water generation device supplies the hypochlorous acid water to the water storage unit in the first treatment.

Thus, in the space cleaning system, the hypochlorous acid can be stably imparted from the space cleaning device described above using the hypochlorous acid water supplied from the hypochlorous acid water generation device. In other words, it is possible to provide a space cleaning system capable of stably imparting the hypochlorous acid in a case where the micronization operation of including the hypochlorous acid in the micronized water and releasing the hypochlorous acid is continuously performed.

Further, in the space cleaning system according to the present disclosure, the space cleaning device is one of the plurality of space cleaning devices, and the plurality of space cleaning devices includes a first space cleaning device that is a space cleaning device and a second space cleaning device different from the first space cleaning device. The hypochlorous acid water generation device is connected to a plurality of space cleaning devices installed in a predetermined target space, being able to supply hypochlorous acid water, and the first space cleaning device and the second space cleaning device are controlled, operation start timings of the humidifying and cleaning unit after the first treatment being different from each other.

In this way, a variation range of the concentration of the hypochlorous acid contained in the air in the predetermined target space can be reduced by the hypochlorous acid released from the humidifying and cleaning unit of the first space cleaning device and the hypochlorous acid released from the humidifying and cleaning unit of the second space cleaning device. In other words, it is possible to provide a space cleaning system capable of stabilizing the concentration of the hypochlorous acid contained in the air in the predetermined target space in a case where the micronization operation of including the hypochlorous acid in the micronized water and releasing the hypochlorous acid is continuously performed.

Further, the space cleaning device according to the present disclosure includes: the humidifying and cleaning unit that generates the hypochlorous acid water micronized by the micronization operation of centrifugally crushing and micronizing the hypochlorous acid water pumped from the water storage unit by the rotation of the pumping pipe, and includes and releases the micronized hypochlorous acid water in the air flowing inside; and the controller that controls the micronization operation. The controller is configured to perform a wastewater treatment of the hypochlorous acid water stored in the water storage unit during the micronization operation based on the time information specified in advance, that is, the time information until the content of the hypochlorous acid contained in the hypochlorous acid water stored in the water storage unit becomes less than or equal to the reference content.

Thus, based on the time information, the content of the hypochlorous acid contained in the hypochlorous acid water stored in the water storage unit is maintained higher than the reference content. Therefore, the hypochlorous acid of the set concentration can be stably imparted to the air released from the humidifying and cleaning unit.

Hereinafter, exemplary embodiments of the present disclosure are described with reference to the accompanying drawings. Note that the following exemplary embodiments are examples embodying the present disclosure, and do not limit the technical scope of the present disclosure. Further, throughout the drawings, the same parts are denoted by the same reference numerals, and duplicated description thereof is omitted. Furthermore, details of each part not directly related to the present disclosure are not described for each drawing in order to avoid duplication.

### First Exemplary Embodiment

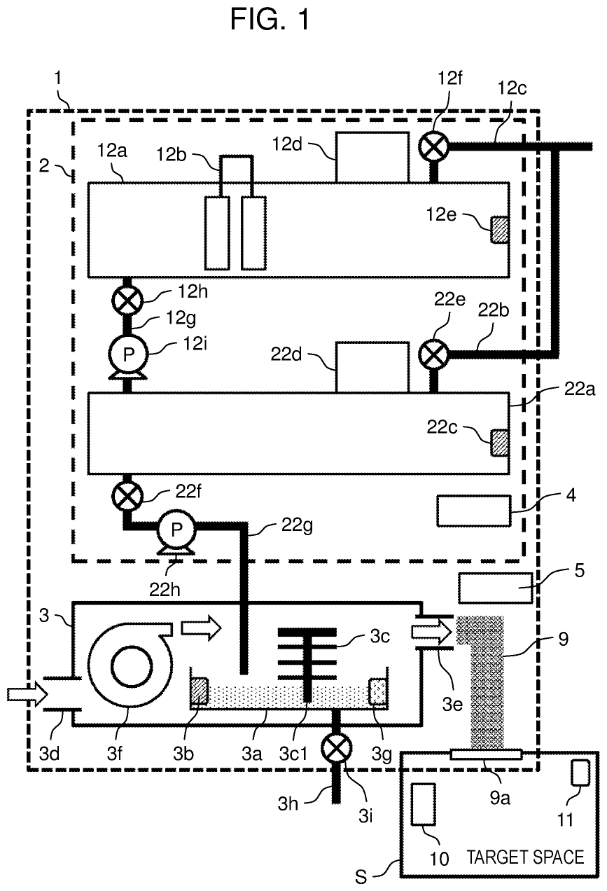

Space cleaning system 1 according to a first exemplary embodiment of the present disclosure is described with reference to FIG. 1. FIG. 1 is a schematic diagram of space cleaning system 1 according to the first exemplary embodiment of the present disclosure.

Space cleaning system 1 according to the first exemplary embodiment of the present disclosure includes: hypochlorous acid water generation device 2 that generates the hypochlorous acid water by electrolyzing a chloride aqueous solution; and humidifying and cleaning device 3 that generates the hypochlorous acid water micronized by the micronization operation of micronizing the hypochlorous acid water supplied from hypochlorous acid water generation device 2 by a centrifugal crushing method, and causes air flowing inside humidifying and cleaning device 3 to contain and release the micronized hypochlorous acid water.

In space cleaning system 1, the air (air containing water and the hypochlorous acid) released from humidifying and cleaning device 3 is supplied to target space S (for example, an indoor space) to sterilize and deodorize target space S. At this time, in space cleaning system 1, based on the concentration (content) of the hypochlorous acid contained in the hypochlorous acid water stored in humidifying and cleaning device 3, a control is performed for draining the hypochlorous acid water stored in the device and supplying new hypochlorous acid water. Thus, space cleaning system 1 can stably impart the air containing the hypochlorous acid to target space S. Details are described later.

As shown in FIG. 1, space cleaning system 1 mainly includes hypochlorous acid water generation device 2 as well as humidifying and cleaning device 3.

<Hypochlorous Acid Water Generation Device>

First, a configuration of hypochlorous acid water generation device 2 is described.

Hypochlorous acid water generation device 2 is a device for generating the hypochlorous acid water by electrolyzing the chloride aqueous solution to be an electrolyte. Specifically, as shown in FIG. 1, hypochlorous acid water generation device 2 includes electrolyzer 12a, dilution tank 22a, first water supply pipe 12g, first water stop valve 12h, first pump 12i, second water supply pipe 22g, second water stop valve 22f, second pump 22h, and hypochlorous acid controller 4.

Electrolyzer 12a is a bath for generating the hypochlorous acid water by electrolysis of the chloride aqueous solution to be an electrolyte. Specifically, as shown in FIG. 1, electrolyzer 12a includes electrode 12b, first water pipe 12c, chloride ion tank 12d, electrolyzer water level sensor 12e, and first water valve 12f.

In electrolyzer 12a, tap water introduced from first water pipe 12c and a substance containing chloride ions (a chloride chemical) supplied from chloride ion tank 12d are mixed to prepare an aqueous solution containing chloride ions (a chloride aqueous solution), and the chloride aqueous solution is electrolyzed by an action of electrode 12b to generate the hypochlorous acid water.

Hereinafter, each component of electrolyzer 12a is described.

Electrode 12b is a member for electrolyzing the aqueous solution containing chloride ions such as saline. Electrode 12b includes a pair of an anode and a cathode, and has a catalyst film on a surface of a conductive substrate. For example, titanium, tantalum, nickel, stainless steel, or the like is used for the conductive substrate, and titanium having high corrosion resistance against the hypochlorous acid is preferable. Further, as the catalyst contained in the catalyst film, for example, iridium, a platinum group metal, or the like is used. Thus, an electrolysis reaction at electrode 12b can be activated. A plurality of electrodes 12b may be provided according to a size of electrolyzer 12a or the amount of the hypochlorous acid water to be generated.

First water pipe 12c is a pipe for introducing the tap water from outside of space cleaning system 1 into electrolyzer 12a. One end of first water pipe 12c is connected to electrolyzer 12a, and the other end is connected to a water supply facility (not illustrated).

Chloride ion tank 12d is a container for accommodating the substance containing chloride ions (the chloride chemical) to be supplied to electrolyzer 12a. The substance containing chloride ions is an electrolyte capable of generating the hypochlorous acid water, and is not particularly limited as long as it contains the chloride ions even in a small amount, and examples thereof include powders such as sodium chloride, calcium chloride and magnesium chloride, as well as tablet-shaped solids. Further, the substance containing chloride ions may be, for example, an aqueous solution where sodium chloride or the like is dissolved or a liquid such as hydrochloric acid.

In addition, in a case where the substance containing chloride ions is accommodated as a liquid, the substance may be held as an aqueous solution having a higher concentration than the concentration of chloride ions during electrolysis in electrolyzer 12a. Thus, chloride ion tank 12d can be downsized, and a frequency where a user replenishes the substance containing the chloride ions to chloride ion tank 12d can be reduced.

Further, chloride ion tank 12d may include a mechanism for supplying the substance containing chloride ions to electrolyzer 12a. For example, as a mechanism for supplying a tablet of sodium chloride, there is a mechanism where a rotating body partially having a hole and a plate partially having a hole provided under the rotating body are provided below chloride ion tank 12d, and when the rotating body rotates, the tablet dropped in the hole of the rotating body drops from the hole opened in the plate. Further, examples of the mechanism for supplying the hydrochloric acid include a mechanism for passing water by opening and closing an electromagnetic valve, a pump, and the like.

Electrolyzer water level sensor 12e is a member that is installed at a predetermined position in electrolyzer 12a and detects the water level of tap water or the hypochlorous acid water in electrolyzer 12a. Electrolyzer water level sensor 12e is communicably connected to hypochlorous acid controller 4 in a wireless or wired manner, detects whether a prescribed amount of tap water has been introduced into electrolyzer 12a, and outputs the detected information to hypochlorous acid controller 4. In addition, electrolyzer water level sensor 12e is used as a means for detecting amount of water in electrolyzer 12a, and may not detect the water level as long as it includes a means for detecting the amount of water in electrolyzer 12a.

First water valve 12f is provided in first water pipe 12c. First water valve 12f is communicably connected to hypochlorous acid controller 4 in a wireless or wired manner, and is opened and closed by a signal received from hypochlorous acid controller 4. Thus, it is possible to introduce or stop the tap water into electrolyzer 12a. As first water valve 12f, an electromagnetic valve can be used.

Electrolyzer 12a is configured by the components above.

First water supply pipe 12g for supplying the hypochlorous acid water in the tank to dilution tank 22a is provided on a bottom surface of a housing constituting electrolyzer 12a. Here, a bottom surface of electrolyzer 12a may be a flat surface (a surface substantially parallel to a floor surface), but is preferably inclined toward first water supply pipe 12g in order to efficiently supply the hypochlorous acid water in electrolyzer 12a to dilution tank 22a without waste.

Assuming that the hypochlorous acid water cannot be supplied to dilution tank 22a due to a factor such as a failure of first water stop valve 12h or water in electrolyzer 12a being washed, electrolyzer 12a may be provided with a water distribution means such as a drain port and a drain pump. Furthermore, electrolyzer 12a may be provided with a stirring means such as a circulation pump or a stirring blade in order to equalize chloride ion concentration or hypochlorous acid concentration in the electrolyzer.

First water supply pipe 12g is a pipe that communicably connects electrolyzer 12a and dilution tank 22a and supplies the hypochlorous acid water generated in electrolyzer 12a to dilution tank 22a. First water supply pipe 12g includes first water stop valve 12h, and can block the supply of the hypochlorous acid water from electrolyzer 12a to dilution tank 22a, prevent the backflow of the hypochlorous acid water from dilution tank 22a to electrolyzer 12a, and prevent the gas generated in dilution tank 22a from entering electrolyzer 12a.

First water stop valve 12h is provided in first water supply pipe 12g. First water stop valve 12h is communicably connected to hypochlorous acid controller 4 in a wireless or wired manner, and is opened and closed by the signal received from hypochlorous acid controller 4. As first water stop valve 12h, an electromagnetic valve can be used.

First pump 12i is provided in first water supply pipe 12g. First pump 12i is a device that causes the hypochlorous acid water to flow through first water supply pipe 12g in a state where first water stop valve 12h is “opened” when the hypochlorous acid water is supplied from electrolyzer 12a to dilution tank 22a. First pump 12i is communicably connected to hypochlorous acid controller 4 in a wireless or wired manner, and operates according to the signal received from hypochlorous acid controller 4. When first water stop valve 12h and first pump 12i operate in conjunction with each other, it is possible to introduce or stop the hypochlorous acid water supplied from electrolyzer 12a into dilution tank 22a.

Next, dilution tank 22a is described.

Dilution tank 22a is a tank that is installed below electrolyzer 12a (downward in a vertical direction) and is used for supplying water to humidifying and cleaning device 3 outside the device by diluting the hypochlorous acid water generated in electrolyzer 12a with tap water and adjusting a hydrogen ion concentration index (pH). Specifically, as shown in FIG. 1, dilution tank 22a includes second water pipe 22b, dilution tank water level sensor 22c, pH adjusting agent tank 22d, and second water valve 22e.

Dilution tank 22a mixes a certain amount of hypochlorous acid water introduced from electrolyzer 12a and tap water introduced from second water pipe 22b to dilute the hypochlorous acid water. Further, dilution tank 22a dissolves and mixes the pH adjusting agent supplied from pH adjusting agent tank 22d to adjust the pH of the hypochlorous acid water, and supplies the hypochlorous acid water to humidifying and cleaning device 3 by second pump 22g. In other words, the pH adjusting agent for adjusting the pH of hypochlorous acid water is added to the hypochlorous acid water stored in humidifier tank 3a of humidifying and cleaning device 3. Then, after the water is supplied to humidifying and cleaning device 3, dilution tank 22a dilutes and generates again the hypochlorous acid water and stands by.

Hereinafter, each component of dilution tank 22a is described.

Second water pipe 22b is a pipe for introducing tap water from outside of space cleaning system 1 into dilution tank 22a.

Second water pipe 22b has one end connected to dilution tank 22a and the other end connected to a water supply facility (not illustrated) via first water pipe 12c. Second water pipe 22b can also be said to be a pipe branched from first water pipe 12c.

Dilution tank water level sensor 22c is a member that is installed at a predetermined position in dilution tank 22a and detects the water level of tap water or the hypochlorous acid water in dilution tank 22a. Dilution tank water level sensor 22c is communicably connected to hypochlorous acid controller 4 in a wireless or wired manner, detects whether the prescribed amount of tap water has been introduced into dilution tank 22a, and outputs the detected information to hypochlorous acid controller 4.

Dilution tank water level sensor 22c detects whether or not the hypochlorous acid water in dilution tank 22a has been supplied to the outside of the device, and outputs the detected information to hypochlorous acid controller 4. In addition, dilution tank water level sensor 22c is used as a means for detecting the amount of water in dilution tank 22a, and may not detect the water level as long as it includes a means for detecting the amount of water in dilution tank 22a.

pH adjusting agent tank 22d is a container for accommodating the pH adjusting agent to be supplied to dilution tank 22a. The pH adjusting agent is a substance capable of adjusting the pH of the hypochlorous acid water, and examples thereof include powders such as phosphate, acetate, carbonate, citric acid, tartaric acid, hydroxide, or ammonium salt, or tablet-shaped solids. Further, the pH adjusting agent may be, for example, an aqueous solution where a phosphate or the like is dissolved, or a liquid such as hydrochloric acid, sulfuric acid, nitric acid, acetic acid, or phosphoric acid.

In addition, in a case where the pH adjusting agent is accommodated in a liquid, the pH adjusting agent may be accommodated as an aqueous solution having a higher concentration than the pH adjusting agent concentration to be supplied to dilution tank 22a. Thus, pH adjusting agent tank 22d can be downsized, and the frequency where the user replenishes the pH adjusting agent can be reduced.

Further, pH adjusting agent tank 22d may include a mechanism for supplying the pH adjusting agent to dilution tank 22a. For example, as the mechanism for supplying the tablet of a phosphate buffer, there is the mechanism where the rotating body partially having a hole and the plate partially having a hole provided under the rotating body are provided below pH adjusting agent tank 22d, and when the rotating body rotates, the tablet dropped into the hole of the rotating body drops from the hole opened in the plate. Further, examples of a mechanism for supplying the aqueous solution where the phosphate or the like is dissolved include a mechanism for passing water by opening and closing an electromagnetic valve, a pump, and the like. In addition, a pH adjusting method may be a method of blowing gas such as carbon dioxide gas into the hypochlorous acid water in dilution tank 22a.

Second water valve 22e is provided in second water pipe 22b. Second water valve 22e is communicably connected to hypochlorous acid controller 4 in a wireless or wired manner, and is opened and closed by the signal received from hypochlorous acid controller 4. Thus, it is possible to introduce or stop the tap water into dilution tank 22a. An electromagnetic valve can be used as second water valve 22e. In addition, as long as second pump 22g has a water-blocking property, second water stop valve 22f is not necessarily required.

Dilution tank 22a is configured by the components above.

Second water supply pipe 22g for supplying the hypochlorous acid water in the tank to humidifying and cleaning device 3 is provided on a bottom surface of a housing constituting dilution tank 22a. Here, a bottom surface of dilution tank 22a may be a flat surface (a surface substantially parallel to the floor surface), but is preferably inclined toward second water supply pipe 22g in order to efficiently supply the hypochlorous acid water in dilution tank 22a to humidifying and cleaning device 3 without waste.

Assuming a case where the hypochlorous acid water cannot be supplied to humidifying and cleaning device 3 due to a factor such as a failure of second water stop valve 22f or a case where water cleaning in dilution tank 22a is performed, dilution tank 22a may be provided with a water distributing means such as a drain port and a drain pump. Furthermore, in order to equalize the hypochlorous acid water concentration or the pH adjusting agent concentration in dilution tank 22a, the stirring means such as the circulation pump or the stirring blade may be provided.

Second water supply pipe 22g is a pipe that communicably connects dilution tank 22a and humidifying and cleaning device 3 and supplies the hypochlorous acid water diluted and pH adjusted in dilution tank 22a to humidifying and cleaning device 3. Second water supply pipe 22g includes second water stop valve 22f, and can block supply of the hypochlorous acid water from electrolyzer 12a to dilution tank 22a.

Second water stop valve 22f is provided in second water supply pipe 22g. Second water stop valve 22f is communicably connected to hypochlorous acid controller 4 in a wireless or wired manner, and is opened and closed by the signal received from hypochlorous acid controller 4. An electromagnetic valve can be used as second water stop valve 22f.

Second pump 22h is provided in second water supply pipe 22g. Second pump 22h is a device that causes the hypochlorous acid water to flow through second water supply pipe 22g in a state where second water stop valve 22f is “opened” when the hypochlorous acid water is supplied from electrolyzer 12a to dilution tank 22a. Second pump 22h is communicably connected to hypochlorous acid controller 4 in a wireless or wired manner, and operates according to the signal received from hypochlorous acid controller 4. When second water supply pipe 22g and second pump 22h operate in conjunction with each other, it is possible to introduce or stop the hypochlorous acid water supplied from dilution tank 22a into humidifying and cleaning device 3.

<Humidifying and Cleaning Device>

Next, a configuration of humidifying and cleaning device 3 is described.

Humidifying and cleaning device 3 is a device that generates the hypochlorous acid water micronized by the micronization operation of micronizing the hypochlorous acid water through centrifugal crushing, causes air flowing inside humidifying and cleaning device 3 to contain and release the micronized hypochlorous acid water to clean target space S. Specifically, as shown in FIG. 1, humidifying and cleaning device 3 includes humidifier tank 3a, humidifier tank water level sensor 3b, centrifugal crushing unit 3c, air introduction port 3d, air delivery port 3e, blower 3f, hypochlorous acid water concentration sensor 3g, drain pipe 3h, and drain valve 3i. In addition, humidifying and cleaning device 3 corresponds to a “space cleaning device” in the claims.

Hereinafter, each component of humidifying and cleaning device 3 is described.

Humidifier tank 3a is a water storage container for storing the hypochlorous acid water supplied from hypochlorous acid water generation device 2 (dilution tank 22a). In addition, humidifier tank 3a corresponds to a “water storage unit” in the claims.

Humidifier tank water level sensor 3b is a member that is installed at a predetermined position in humidifier tank 3a and detects the water level of the hypochlorous acid water supplied from hypochlorous acid water generation device 2. Humidifier tank water level sensor 3b is communicably connected to humidification controller 5 in a wireless or wired manner, detects the water level of humidifier tank 3a, and outputs the detected information to humidification controller 5. In addition, humidifier tank water level sensor 3b is used as a means for detecting the amount of water in humidifier tank 3a, and may not detect the water level as long as it includes a means for detecting the amount of water in humidifier tank 3a.

Centrifugal crushing unit 3c is a member for allowing the air introduced into humidifying and cleaning device 3 to contain moisture. When pumping pipe 3c1 included in centrifugal crushing unit 3c rotates at a high speed, centrifugal crushing unit 3c sucks (pumps) water (the hypochlorous acid water) in humidifier tank 3a by a centrifugal force, discharges the sucked water from the centrifugal plate in the surroundings (a centrifugal direction) to collide with (centrifugally crush) a crushing wall, and micronizes water particles. At this time, the hypochlorous acid is added to the air passing through centrifugal crushing unit 3c together with the micronized water. Further, centrifugal crushing unit 3c is communicably connected to humidification controller 5 in a wireless or wired manner, and operates according to a signal received from humidification controller 5. In addition, centrifugal crushing unit 3c corresponds to a “humidifying and cleaning unit” in the claims.

Air introduction port 3d is an opening for introducing the air in target space S (for example, an indoor space) into humidifying and cleaning device 3. Air introduction port 3d is communicably connected to a suction port (not illustrated) provided in target space S via a duct (not illustrated).

Air delivery port 3e is an opening for discharging the air humidified by an action of centrifugal crushing unit 3c to target space S outside humidifying and cleaning device 3. Air delivery port 3e is communicably connected to blow-out port 9a provided in target space S via duct 9.

Blower 3f is a member that introduces air into humidifying and cleaning device 3 from air introduction port 3d and generates a flow for discharging the air humidified by the action of centrifugal crushing unit 3c to the outside of humidifying and cleaning device 3 from air delivery port 3e.

Hypochlorous acid water concentration sensor 3g is a member that is installed at a predetermined position in humidifier tank 3a and detects the concentration (content) of the hypochlorous acid contained in the hypochlorous acid water stored in humidifier tank 3a. Hypochlorous acid water concentration sensor 3g is communicably connected to humidification controller 5 in a wireless or wired manner, detects the concentration (content) of the hypochlorous acid contained in the hypochlorous acid water stored in humidifier tank 3a, and outputs the detected information to humidification controller 5.

Drain pipe 3h is provided on the bottom surface of humidifier tank 3a, and is a pipe for draining the hypochlorous acid water in humidifier tank 3a to the outside of humidifying and cleaning device 3. Drain pipe 3h includes drain valve 3i, and can block drainage of the hypochlorous acid water from the inside of humidifying and cleaning device 3 to the outside of humidifying and cleaning device 3.

Drain valve 3i is provided in drain pipe 3h. Drain valve 3i is communicably connected to humidification controller 5 in a wireless or wired manner, and is opened and closed by the signal received from humidification controller 5. An electromagnetic valve can be used as drain valve 3i.

Humidifying and cleaning device 3 is configured by the components above.

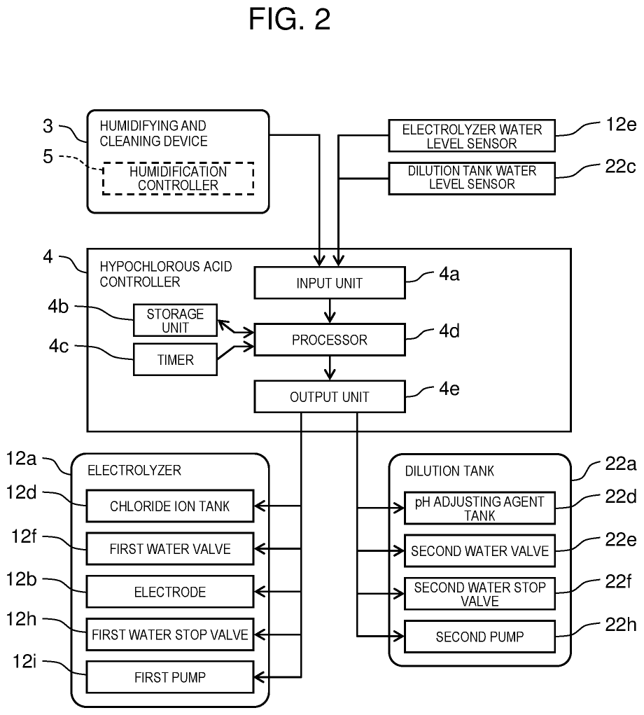

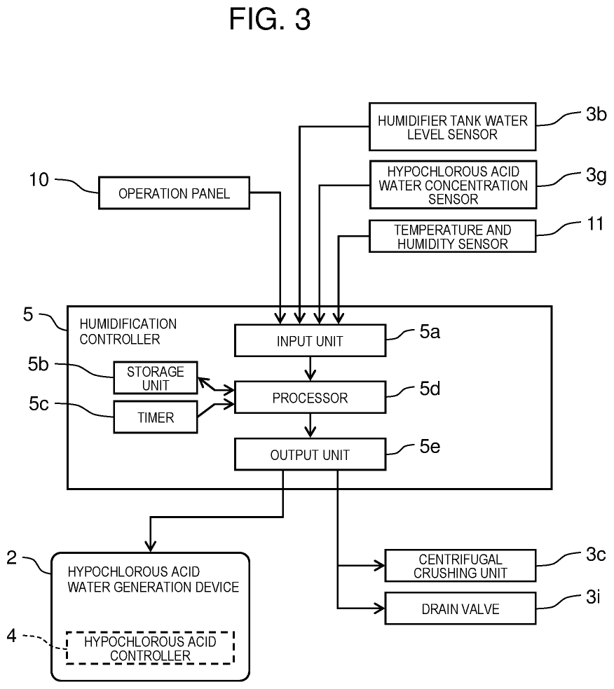

Next, hypochlorous acid controller 4 of hypochlorous acid water generation device 2 and humidification controller 5 of humidifying and cleaning device 3 in space cleaning system 1 are described with reference to FIGS. 2 and 3. FIG. 2 is a block diagram showing the configuration of hypochlorous acid controller 4 of hypochlorous acid water generation device 2 in space cleaning system 1. FIG. 3 is a block diagram illustrating the configuration of humidification controller 5 of humidifying and cleaning device 3 in space cleaning system 1.

Here, hypochlorous acid controller 4 and humidification controller 5 have a computer system including a processor and a memory. Then, with the processor executing a program stored in the memory, the computer system functions as the controller. Here, the program executed by the processor is recorded in advance in the memory of the computer system, but may be provided by being recorded in a non-transitory recording medium such as a memory card, or may be provided through a telecommunication line such as the Internet.

<Hypochlorous Acid Controller of Hypochlorous Acid Water Generation Device>

First, hypochlorous acid controller 4 of hypochlorous acid water generation device 2 is described.

Hypochlorous acid controller 4 controls a processing operation in hypochlorous acid water generation device 2. Here, the processing operation includes an operation related to an electrolysis processing in electrolyzer 12a, an operation related to a dilution processing and a pH adjustment processing in dilution tank 22a, and an operation related to a supply processing of the hypochlorous acid water to humidifying and cleaning device 3.

Specifically, as shown in FIG. 2, hypochlorous acid controller 4 includes input unit 4a, storage unit 4b, timer 4c, processor 4d, and output unit 4e.

<Operation Related to Electrolysis Processing in Electrolyzer>

Hypochlorous acid controller 4 performs the following processing as an operation related to the electrolysis processing in electrolyzer 12a.

Input unit 4a receives information regarding time received from timer 4c as a trigger of the electrolysis processing of electrolyzer 12a, and outputs the information to processor 4d.

Processor 4d specifies control information based on the information regarding the time received from timer 4c and setting information received from storage unit 4b, and outputs the control information to output unit 4e. Here, the setting information includes information regarding a start time or an end time of the hypochlorous acid water generation, information regarding a supply amount of tap water to be introduced into electrolyzer 12a, information regarding an input amount of the substance containing chloride ions in chloride ion tank 12d, information regarding electrolysis conditions (time, current value, voltage, and the like) in electrode 12b, information regarding an opening or closing timing of first water valve 12f, information regarding an opening or closing timing of first water valve 12f and first water stop valve 12h, and information regarding an on or off operation of first pump 12i.

The electrolysis conditions in electrode 12b can be determined from the amount of tap water in electrolyzer 12a, the chloride ion concentration, the electrolysis time, and a degree of deterioration of electrode 12b, and an algorithm is created and set, and stored in storage unit 4b.

Output unit 4e outputs a signal (a control signal) to each device (electrode 12b, chloride ion tank 12d, first water valve 12f, and first water stop valve 12h) based on the received control information.

More specifically, to start with, first water stop valve 12h maintains a closed state based on the signal received from output unit 4e. First pump 12i maintains a stopped state based on the signal received from output unit 4e.

Then, first water valve 12f is opened based on the signal received from output unit 4e. Thus, supply of tap water from first water pipe 12c to electrolyzer 12a is started. Thereafter, first water valve 12f is closed based on a signal transmitted from output unit 4e that has received water level information (full water) from electrolyzer water level sensor 12e. Thus, electrolyzer 12a is in a state where tap water is supplied at a set supply amount.

Next, chloride ion tank 12d starts its operation based on the signal received from output unit 4e, and inputs a substance containing a predetermined amount of chloride ions into electrolyzer 12a to stop. Thus, the substance containing chloride ions is dissolved in the tap water. Therefore, electrolyzer 12a is in a state where the aqueous solution containing chloride ions (the chloride aqueous solution) is generated.

Then, electrode 12b starts electrolysis of the chloride aqueous solution based on the signal received from output unit 4e, and generates and stops the hypochlorous acid water under set conditions. The hypochlorous acid water generated by electrode 12b has, for example, the hypochlorous acid concentration of 100 ppm to 150 ppm (for example, 120 ppm) and a pH of 7 to 8.5 (for example, 8.0).

As described above, hypochlorous acid controller 4 causes electrolyzer 12a to perform the electrolysis processing.

<Operation Related to Dilution Processing and pH Adjustment Processing in Dilution Tank>

Hypochlorous acid controller 4 executes the following processing as an operation related to the dilution processing and the pH adjustment processing in dilution tank 22a.

Input unit 4a receives the water level information received from dilution tank water level sensor 22c as a trigger of the dilution processing of dilution tank 22a, and outputs the water level information to processor 4d.

Processor 4d specifies control information based on the information regarding the time received from timer 4c and setting information received from storage unit 4b, and outputs the control information to output unit 4e. Here, the setting information includes information regarding supply amount of the hypochlorous acid water received from electrolyzer 12a, information regarding supply amount of the pH adjusting agent in pH adjusting agent tank 22d, information regarding supply amount of tap water to be introduced into dilution tank 22a, information regarding an opening or closing timing of second water valve 22e, first water stop valve 12h, and second water stop valve 22f, and information regarding on or off operations of first pump 12i and second pump 22h.

Here, the input amount of the pH adjusting agent can be determined by the amount and concentration of the hypochlorous acid water introduced from electrolyzer 12a into dilution tank 22a and a target pH of the hypochlorous acid water prepared in dilution tank 22a, and is set by creating an algorithm and stored in storage unit 4b.

Then, output unit 4e outputs the signal (the control signal) to each device (pH adjusting agent tank 22d, second water valve 22e, second water stop valve 22f, and second pump 22h) based on the received control information.

More specifically, first, first water stop valve 12h and second water stop valve 22f maintain a closed state based on the signal received from output unit 4e. First pump 12i and second pump 22h maintain the stopped state based on the signal received from output unit 4e.

Then, second water valve 22e is opened based on the signal received from output unit 4e. Thus, the supply of tap water from second water pipe 22b to dilution tank 22a is started. Thereafter, second water valve 22e is closed based on a signal transmitted from output unit 4e that has received the water level information (the water level of the prescribed amount) from dilution tank water level sensor 22c. Thus, in dilution tank 22a, tap water is supplied at the set supply amount.

Then, first water stop valve 12h is opened based on a signal received from output unit 4e. Then, first pump 12i operates in accordance with the operation of first water stop valve 12h based on the signal received from output unit 4e. Thus, in dilution tank 22a, the supply of the hypochlorous acid water is started from the electrolyzer 12a.

Thereafter, first water valve 12f is closed based on a signal transmitted from output unit 4e that has received the information regarding the time (the required time for supplying the prescribed amount) from timer 4c. First pump 12i is also stopped. Thus, in dilution tank 22a, the hypochlorous acid water is supplied from electrolyzer 12a to the tap water in dilution tank 22a at the set supply amount. Thus, the hypochlorous acid water in dilution tank 22a is diluted.

Subsequently, pH adjusting agent tank 22d starts an operation based on the signal received from output unit 4e, and inputs the predetermined amount of pH adjusting agent into dilution tank 22a and stops operation. Thus, in dilution tank 22a, the pH adjusting agent is dissolved in the diluted hypochlorous acid water, and the hypochlorous acid water whose pH is adjusted is generated. In other words, in dilution tank 22a, the hypochlorous acid water supplied from electrolyzer 12a, tap water supplied from second water pipe 22b, and the pH adjusting agent supplied from pH adjusting agent tank 22d are mixed to generate the hypochlorous acid water under the set conditions (concentration and pH). The mixed and diluted hypochlorous acid water has, for example, a hypochlorous acid concentration of 10 ppm to 50 ppm (for example, 30 ppm) and a pH of 7 to 5 (for example, 6.5).

As described above, hypochlorous acid controller 4 causes dilution tank 22a to execute the dilution processing and the pH adjustment processing.

<Operation related to processing of supplying the hypochlorous acid water to humidifying and cleaning device 3>

Hypochlorous acid controller 4 performs the following processing as an operation related to the processing of supplying the hypochlorous acid water to humidifying and cleaning device 3.

Input unit 4a receives a signal (a water supply request signal to be described later) received from humidification controller 5 of humidifying and cleaning device 3 as a trigger of the hypochlorous acid water supply processing to humidifying and cleaning device 3, and outputs the signal to processor 4d.

Processor 4d specifies control information based on the information regarding the time received from timer 4c and setting information received from storage unit 4b, and outputs the control information to output unit 4e. Here, the setting information includes information regarding the supply amount of the hypochlorous acid water supplied from dilution tank 22a, information regarding the opening or closing timing of second water stop valve 22f, and information regarding an on or off operation of second pump 22h.

Then, output unit 4e outputs a signal (the control signal) to each device (second water stop valve 22f and second pump 22h) based on the received control information.

Next, second water stop valve 22f is opened based on the signal received from output unit 4e. Then, second pump 22h operates in accordance with the operation of second water stop valve 22f based on the signal received from output unit 4e. Thus, in dilution tank 22a, the supply of the hypochlorous acid water to humidifying and cleaning device 3 (humidifier tank 3a) is started.

Thereafter, second water stop valve 22f is closed based on a signal transmitted from output unit 4e that has received the information regarding the time (the required time for supplying the prescribed amount) from timer 4c. Then, second pump 22h also stops. Thus, dilution tank 22a supplies the hypochlorous acid water to humidifying and cleaning device 3 (humidifier tank 3a) at the set supply amount.

As described above, hypochlorous acid controller 4 causes the supply processing of the hypochlorous acid water to humidifying and cleaning device 3 to be executed.

<Humidification Controller of Humidifying and Cleaning Device>

Next, humidification controller 5 of humidifying and cleaning device 3 is described.

Humidification controller 5 controls a processing operation in humidifying and cleaning device 3. Specifically, as shown in FIG. 3, the humidification control unit 5 includes input unit 5a, storage unit 5b, timer 5c, processor 5d, and output unit 5e.

Input unit 5a receives user input information received from operation panel 10, temperature and humidity information of the air in target space S received from temperature and humidity sensor 11, the water level information of the hypochlorous acid water in humidifier tank 3a received from humidifier tank water level sensor 3b, and the concentration information (content information) of the hypochlorous acid contained in the hypochlorous acid water in humidifier tank 3a received from hypochlorous acid water concentration sensor 3g. Input unit 5a outputs all the received information to processor 5d.

Here, operation panel 10 is a terminal where the user input information (for example, air volume, target temperature, target humidity, presence or absence of addition of the hypochlorous acid, target supply amount level of the hypochlorous acid, and the like) related to humidifying and cleaning device 3 is input, and is communicably connected to humidification controller 5 in a wireless or wired manner.

Temperature and humidity sensor 11 is a sensor that is provided in target space S and senses the temperature and humidity of the air in target space S.

Storage unit 5b stores the user input information received by input unit 5a and supply setting information in an operation of supplying the hypochlorous acid to the air flowing inside humidifying and cleaning device 3. Storage unit 5b outputs the stored supply setting information to processor 5d. In addition, the supply setting information in a hypochlorous acid supply operation can also be referred to as humidification setting information in a humidification operation of centrifugal crushing unit 3c.

Timer 5c outputs time information related to the current time to processor 5d.

Processor 5d receives various types of information (user input information, temperature and humidity information, water level information, and concentration information) received from input unit 5a and the supply setting information received from storage unit 5b. Processor 5d specifies control information related to the humidifying and cleaning operation using the received user input information and supply setting information.

Further, in a case where the water level information received from humidifier tank water level sensor 3b includes information on a water level indicating a shortage of the hypochlorous acid water in humidifier tank 3a, processor 5d specifies information (water supply request information) on a water supply request to hypochlorous acid controller 4 of hypochlorous acid water generation device 2.

Further, in a case where the concentration of hypochlorous acid included in the concentration information received from hypochlorous acid water concentration sensor 3g (the concentration of hypochlorous acid contained in the hypochlorous acid water in humidifier tank 3a) is less than or equal to a reference concentration, processor 5d specifies the control information related to the first treatment of draining the hypochlorous acid water stored in humidifier tank 3a and supplying new hypochlorous acid water.

Then, processor 5d specifies information (the water supply request information) related to the water supply request to hypochlorous acid controller 4 of hypochlorous acid water generation device 2. In addition, the reference concentration is set to a minimum concentration necessary for obtaining a sterilization and deodorization effect in target space S.

Then, processor 5d outputs the specified control information and water supply request information to output unit 5e.

Output unit 5e receives the control information from processor 5d. Output unit 5e is electrically connected to centrifugal crushing unit 3c and drain valve 3i of humidifying and cleaning device 3.

Then, output unit 5e outputs a signal (the control signal) for controlling the humidifying and cleaning operation of humidifying and cleaning device 3 based on the received control information.

Further, output unit 5e receives the water supply request information from processor 5d. Output unit 5e is electrically connected to hypochlorous acid controller 4 of hypochlorous acid water generation device 2. Then, output unit 5e outputs a signal (the water supply request signal) to hypochlorous acid controller 4 based on the received water supply request information.

Then, centrifugal crushing unit 3c and drain valve 3i receive signals transmitted from output unit 5e, and a control of respective operations based on the received signals. Further, hypochlorous acid controller 4 of hypochlorous acid water generation device 2 receives the signal transmitted from output unit 5e, and executes an operation related to the supply processing of the hypochlorous acid water to humidifying and cleaning device 3 based on the received signal.

As described above, humidification controller 5 performs the processing of adding the hypochlorous acid to the air flowing through humidifying and cleaning device 3.

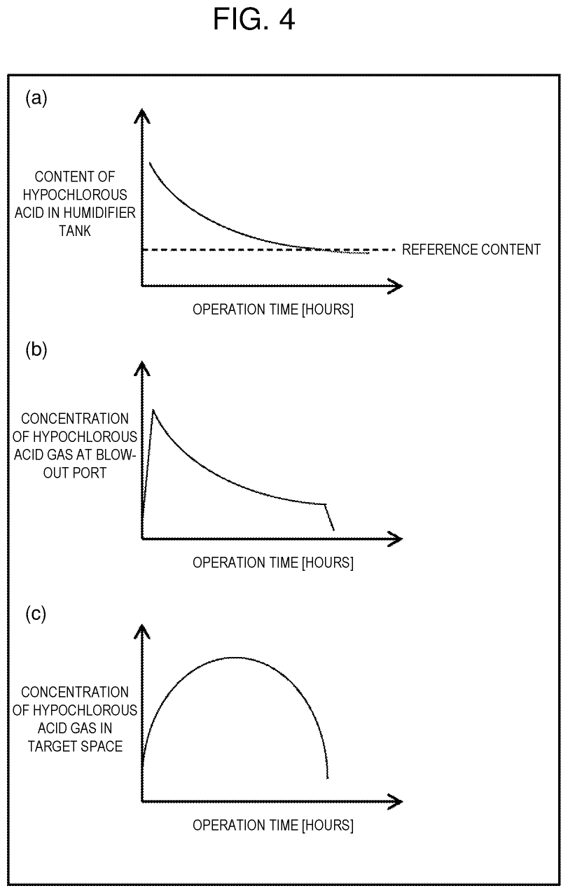

Next, the temporal change in the hypochlorous acid concentration in space cleaning system 1 will be described with reference to FIG. 4. FIG. 4 is a schematic view showing the temporal change in the hypochlorous acid concentration in space cleaning system 1.

More specifically, part (a) of FIG. 4 is a diagram illustrating a state of the temporal change of a concentration (content) of the hypochlorous acid contained in the hypochlorous acid water in humidifier tank 3a. Part (b) of FIG. 4 is a diagram illustrating a state of the temporal change of the concentration of a hypochlorous acid gas contained in the air blown out from blow-out port 9a (air delivery port 3e of humidifying and cleaning device 3). Part (c) of FIG. 4 is a diagram illustrating a state of the temporal change of the concentration of the hypochlorous acid gas contained in the air in target space S.

As shown in part (a) of FIG. 4, in humidifying and cleaning device 3, the concentration (content) of the hypochlorous acid contained in the hypochlorous acid water stored in humidifier tank 3a decreases with operation time. This is presumed to be because the hypochlorous acid is vaporized and applied to the air due to a fact that a vapor pressure of the hypochlorous acid is higher than a vapor pressure of water.

In addition, if the hypochlorous acid is not vaporized, the hypochlorous acid contained in the water is merely consumed together with the water micronized by centrifugal crushing unit 3c, and thus it is presumed that the hypochlorous acid contained in the hypochlorous acid water does not decrease with the operation time.

Then, as shown in part (b) of FIG. 4, in humidifying and cleaning device 3, when the hypochlorous acid water is supplied to humidifier tank 3a, the hypochlorous acid starts to be released together with water by an action of centrifugal crushing unit 3c, and the concentration of the hypochlorous acid gas blown out from blow-out port 9a increases. Then, as the concentration of the hypochlorous acid contained in the hypochlorous acid water in humidifier tank 3a decreases, the concentration of the hypochlorous acid gas blown out from blow-out port 9a also gradually decreases. In other words, it can be said that in humidifying and cleaning device 3, the concentration (content) of the hypochlorous acid contained in the hypochlorous acid water in humidifier tank 3a is reduced, and the concentration of the hypochlorous acid gas contained in the air blown out from blow-out port 9a is also reduced.

Then, as shown in part (c) of FIG. 4, in target space S, when the hypochlorous acid gas is released from blow-out port 9a, the hypochlorous acid gas diffuses into target space S, and the concentration of the hypochlorous acid gas in target space S gradually increases. Then, as the concentration of the hypochlorous acid gas released from blow-out port 9a decreases, the concentration of the hypochlorous acid gas in target space S also gradually decreases. In other words, it can be said that, in humidifying and cleaning device 3, the concentration (content) of the hypochlorous acid contained in the hypochlorous acid water in humidifier tank 3a is reduced, and the concentration of the hypochlorous acid gas contained in the air in target space S is also reduced.

In space cleaning system 1 according to the present exemplary embodiment, hypochlorous acid water concentration sensor 3g detects the concentration (content) of the hypochlorous acid contained in the hypochlorous acid water in humidifier tank 3a at preset time intervals (for example, one minute) based on the change in the concentration of the hypochlorous acid described above. Then, in a case where the detected concentration (the content) of the hypochlorous acid becomes less than or equal to the reference concentration, the first treatment of draining the hypochlorous acid water where the content of the hypochlorous acid has decreased and newly supplying the hypochlorous acid water having the set concentration is executed. Thus, it is suppressed that the concentration (content) of the hypochlorous acid contained in the hypochlorous acid water in humidifier tank 3a decreases with a lapse of time and the hypochlorous acid gas is not released into target space S.

In addition, by executing the first treatment, the humidification operation by centrifugal crushing unit 3c is temporarily stopped. Therefore, as shown in part (b) of FIG. 4, the concentration of the hypochlorous acid gas blown out from blow-out port 9a temporarily decreases.

As described above, according to humidifying and cleaning device 3 according to the first exemplary embodiment and space cleaning system 1 using humidifying and cleaning device 3, the following effects can be obtained.

(1) Humidifying and cleaning device 3 includes centrifugal crushing unit 3c that generates the hypochlorous acid water micronized by the micronization operation of micronizing the hypochlorous acid water stored in humidifier tank 3a, and adds the micronized hypochlorous acid water to the air flowing inside to release the hypochlorous acid water, and a humidification controller 5 that controls the micronization operation. Humidification controller 5 performs the first treatment of draining the hypochlorous acid water stored in humidifier tank 3a and supplying new hypochlorous acid water during the micronization operation based on the information regarding the content of the hypochlorous acid contained in the hypochlorous acid water stored in humidifier tank 3a.

Thus, the hypochlorous acid contained in the hypochlorous acid water stored in humidifier tank 3a is vaporized and reduced, and is replaced with new hypochlorous acid water by the first treatment before the hypochlorous acid is no longer released at the set concentration from centrifugal crushing unit 3c. Therefore, in humidifying and cleaning device 3, the hypochlorous acid can be stably imparted to the air released from centrifugal crushing unit 3c. In other words, it is possible to provide humidifying and cleaning device 3 capable of stably imparting the hypochlorous acid in a case where the micronization operation of including the hypochlorous acid in the micronized water and releasing the hypochlorous acid is continuously performed.

(2) Humidifying and cleaning device 3 includes a hypochlorous acid water concentration sensor 3g that detects the concentration of the hypochlorous acid contained in the hypochlorous acid water stored in centrifugal crushing unit 3c. Humidification controller 5 performs a control to execute the first treatment in a case where the concentration of the hypochlorous acid included in the concentration information detected by hypochlorous acid water concentration sensor 3g is less than or equal to the reference concentration.

Thus, in humidifying and cleaning device 3, since the content of the hypochlorous acid contained in the hypochlorous acid water stored in humidifier tank 3a is maintained higher than the reference concentration based on the concentration information, the hypochlorous acid of the set concentration can be stably imparted to the air released from centrifugal crushing unit 3c.

(3) Space cleaning system 1 includes humidifying and cleaning device 3 described above and a hypochlorous acid water generation device 2 that generates the hypochlorous acid water by electrolyzing the chloride aqueous solution. Hypochlorous acid water generation device 2 supplies the hypochlorous acid water to humidifier tank 3a in the first treatment.

Thus, in space cleaning system 1, the hypochlorous acid can be stably imparted from humidifying and cleaning device 3 described above using the hypochlorous acid water supplied from hypochlorous acid water generation device 2. In other words, it is possible to provide space cleaning system 1 capable of stably imparting the hypochlorous acid in a case where the micronization operation of adding the hypochlorous acid in the micronized water and releasing the hypochlorous acid is continuously performed.

### Second Exemplary Embodiment

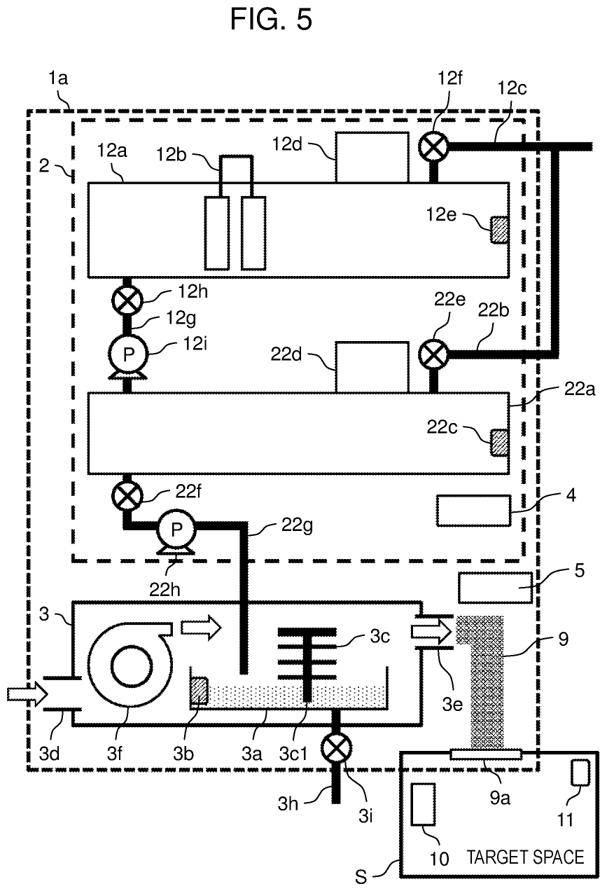

Space cleaning system 1a according to the second exemplary embodiment of the present disclosure will be described with reference to FIG. 5. FIG. 5 is the schematic diagram of space cleaning system 1a according to the second exemplary embodiment of the present disclosure.

Space cleaning system 1a according to the second exemplary embodiment of the present disclosure is different from the first exemplary embodiment in that, humidifying and cleaning device 3 executes the first treatment (processing of draining the hypochlorous acid water stored in humidifier tank 3a and supplying new hypochlorous acid water) at preset time intervals without installing hypochlorous acid water concentration sensor 3g in humidifier tank 3a. Other configurations and control methods of space cleaning system 1a are similar to those of space cleaning system 1 according to the first exemplary embodiment.

In the following, description of the contents already described in the first exemplary embodiment will be omitted as appropriate, and differences from the first exemplary embodiment will be mainly described.

Similarly to space cleaning system 1, space cleaning system 1a includes a hypochlorous acid water generation device 2 that generates the hypochlorous acid water by electrolyzing the chloride aqueous solution, and humidifying and cleaning device 3 that generates the hypochlorous acid water micronized by the micronization operation of micronizing the hypochlorous acid water supplied from hypochlorous acid water generation device 2 by a centrifugal crushing method, and causes air flowing inside humidifying and cleaning device 3 to contain and release the micronized hypochlorous acid water.

Also in space cleaning system 1a, the processing operation is controlled by hypochlorous acid controller 4 of hypochlorous acid water generation device 2 and humidification controller 5 of humidifying and cleaning device 3.

<Hypochlorous Acid Controller of Hypochlorous Acid Water Generation Device>

Since the processing operation (the operation related to the electrolysis processing in electrolyzer 12a, the operation related to the dilution processing and pH adjustment processing in dilution tank 22a, and operation related to the supply processing of the hypochlorous acid water to humidifying and cleaning device 3) of hypochlorous acid water generation device 2 by hypochlorous acid controller 4 of space cleaning system 1a is similar to the processing operation of the exemplary embodiment, the description thereof is omitted.

<Humidification Controller of Humidifying and Cleaning Device>

Humidification controller 5 of space cleaning system 1a controls the processing operation in humidifying and cleaning device 3. Specifically, as shown in FIG. 3, the humidification control unit 5 includes input unit 5a, storage unit 5b, timer 5c, processor 5d, and output unit 5e.

Input unit 5a receives the user input information received from operation panel 10, the temperature and humidity information of the air in target space S received from temperature and humidity sensor 11, and the water level information of the hypochlorous acid water in humidifier tank 3a received from humidifier tank water level sensor 3b. Input unit 5a outputs all the received information to processor 5d.

Storage unit 5b stores the user input information received by input unit 5a and the supply setting information in the operation of supplying the hypochlorous acid to the air flowing in the device. Further, storage unit 5b stores time information (for example, 1 hour) that is specified in response to the temporal change of the hypochlorous acid shown in part (a) of FIG. 4 and until the concentration (content) of the hypochlorous acid contained in the hypochlorous acid water in humidifier tank 3a becomes less than or equal to a preset reference concentration (the reference content). Storage unit 5b outputs the stored supply setting information to processor 5d.

In addition, the time information is time from a start of the micronization operation until the content of the hypochlorous acid becomes less than or equal to the reference content, and is information related to a time estimated in advance by experimental evaluation based on the temporal change of the hypochlorous acid shown in part (a) of FIG. 4. Further, the reference concentration is set to a minimum concentration necessary for obtaining a sterilization and deodorization effect in target space S. Further, the time information is preferably estimated for each concentration of the hypochlorous acid water to be used.

Timer 5c outputs time information related to the current time to processor 5d.

Processor 5d receives various types of information (the user input information and the temperature and humidity information) received from input unit 5a, as well as the supply setting information and the time information received from storage unit 5b. Processor 5d specifies control information related to the humidifying and cleaning operation using the received user input information, supply setting information, and time information.

Further, in a case where the water level information received from humidifier tank water level sensor 3b includes information on a water level indicating a shortage of the hypochlorous acid water in humidifier tank 3a, processor 5d specifies information (water supply request information) on a water supply request to hypochlorous acid controller 4 of hypochlorous acid water generation device 2.

Furthermore, processor 5d specifies control information related to the first treatment of draining the hypochlorous acid water stored in humidifier tank 3a every hour and supplying new hypochlorous acid water based on the time information. Then, based on the time information, processor 5d specifies information (the water supply request information) on the water supply request to hypochlorous acid controller 4 of hypochlorous acid water generation device 2 every hour.

Then, processor 5d outputs the specified control information and water supply request information to output unit 5e.

Output unit 5e receives the control information transmitted from processor 5d. Output unit 5e is electrically connected to centrifugal crushing unit 3c and drain valve 3i of humidifying and cleaning device 3. Then, output unit 5e outputs a signal (the control signal) for controlling the humidifying and cleaning operation of humidifying and cleaning device 3 based on the received control information.

Further, output unit 5e receives the water supply request information transmitted from processor 5d. Output unit 5e is electrically connected to hypochlorous acid controller 4 of hypochlorous acid water generation device 2. Then, output unit 5e transmits a signal (the water supply request signal) to hypochlorous acid controller 4 based on the received water supply request information.

Then, centrifugal crushing unit 3c and drain valve 3i receive signals transmitted from output unit 5e, and execute a control of the respective operations based on the received signals. Further, hypochlorous acid controller 4 receives a signal transmitted from output unit 5e, and executes a control of the water supply operation for humidifying and cleaning device 3 based on the received signal.

As described above, humidification controller 5 of space cleaning system 1a executes the hypochlorous acid imparting processing in humidifying and cleaning device 3.

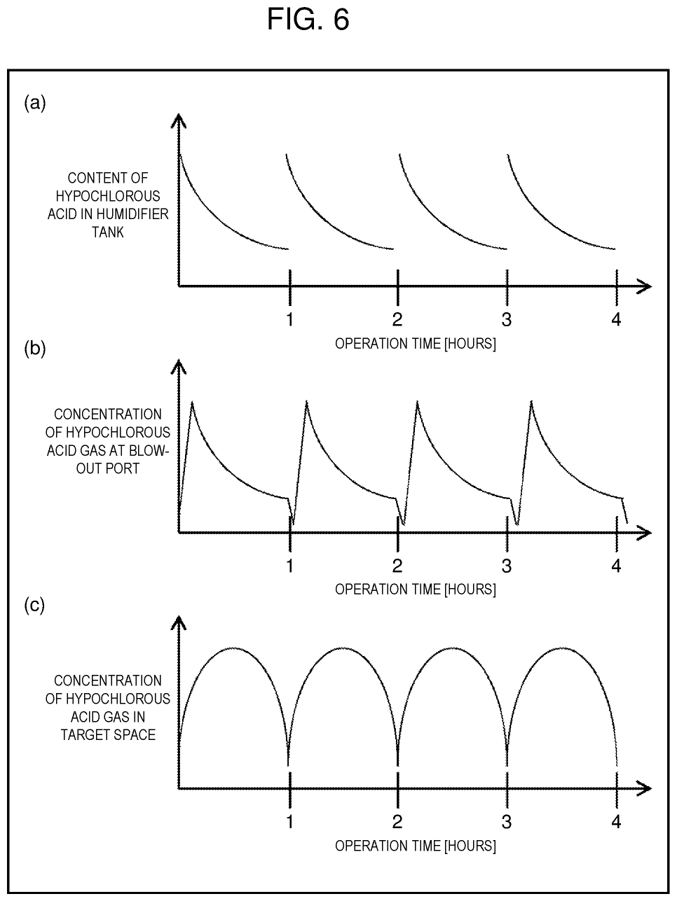

Next, the temporal change in the hypochlorous acid concentration in space cleaning system 1a will be described with reference to FIG. 6. FIG. 6 is a schematic view showing the temporal change in the hypochlorous acid concentration in space cleaning system 1a.

More specifically, part (a) of FIG. 6 is a view showing a state of the temporal change of a concentration (content) of the hypochlorous acid contained in the hypochlorous acid water in humidifier tank 3a. Part (b) of FIG. 6 is a diagram illustrating a state of the temporal change of the concentration of the hypochlorous acid gas contained in the air blown out from blow-out port 9a (air delivery port 3e of humidifying and cleaning device 3). Part (c) of FIG. 6 is a diagram illustrating a state of the temporal change of the concentration of the hypochlorous acid gas contained in the air in target space S.

In humidifying and cleaning device 3 of space cleaning system 1a, as shown in part (a) of FIG. 6, the concentration (content) of the hypochlorous acid contained in the hypochlorous acid water stored in humidifier tank 3a repeats increase and decrease with the operation time every hour. Here, the decrease in the concentration (content) of the hypochlorous acid is due to a reason described with reference to part (a) of FIG. 4, and the increase in the concentration (content) of the hypochlorous acid is due to a replacement with new hypochlorous acid water.

Then, as shown in part (b) of FIG. 6, in humidifying and cleaning device 3 of space cleaning system 1a, the concentration of the hypochlorous acid gas blown out from blow-out port 9a is also repeatedly increased and decreased in response to the increase and decrease of the concentration of the hypochlorous acid contained in the hypochlorous acid water in humidifier tank 3a.

Then, as shown in part (c) of FIG. 6, in target space S, the concentration (content) of the hypochlorous acid contained in the hypochlorous acid water in humidifier tank 3a is repeatedly increased and decreased, and the concentration of the hypochlorous acid gas contained in the air in target space S is also repeatedly decreased and increased.

In space cleaning system 1a according to the second exemplary embodiment, the first treatment (processing of draining the hypochlorous acid water stored in humidifier tank 3a and supplying new hypochlorous acid water) is executed at preset time intervals (for example, 1 hour) without installing hypochlorous acid water concentration sensor 3g in humidifier tank 3a. Thus, the concentration (content) of the hypochlorous acid contained in the hypochlorous acid water in humidifier tank 3a decreases, and it is possible to continuously suppress the hypochlorous acid gas from not being released to target space S.

As described above, according to humidifying and cleaning device 3 in space cleaning system 1a according to the second exemplary embodiment, the following effects can be obtained.

(1) Humidifying and cleaning device 3 in space cleaning system 1a includes centrifugal crushing unit 3c that generates the hypochlorous acid water micronized by the micronization operation for micronizing the hypochlorous acid water stored in humidifier tank 3a, and causes air flowing inside to contain and release the micronized hypochlorous acid water, and a humidification controller 5 that controls the micronization operation. Humidification controller 5 performs the first treatment of draining the hypochlorous acid water stored in humidifier tank 3a and supplying new hypochlorous acid water based on the time information specified in advance and the time information (for example, one hour) from the start of the micronization operation until the content of the hypochlorous acid becomes less than or equal to the reference content.

Thus, the hypochlorous acid contained in the hypochlorous acid water stored in humidifier tank 3a is vaporized and reduced based on the time information specified in advance, and is replaced with new hypochlorous acid water by the first treatment before the hypochlorous acid is no longer released at the set concentration from centrifugal crushing unit 3c. In other words, in humidifying and cleaning device 3, since the content of the hypochlorous acid contained in the hypochlorous acid water stored in humidifier tank 3a is maintained higher than the reference content based on the time information, the hypochlorous acid of the set concentration can be stably imparted to the air released from the centrifugal crushing unit 3g.

(2) In humidifying and cleaning device 3, the centrifugal crushing unit 3g generates the hypochlorous acid water micronized by the micronization operation of centrifugally crushing and micronizing the hypochlorous acid water pumped from humidifier tank 3a by the rotation of pumping pipe 3c1.

Thus, the hypochlorous acid water can be efficiently micronized.

(3) In humidifying and cleaning device 3, the time information is specified in advance for each concentration of the hypochlorous acid water stored in humidifier tank 3a.

Thus, in humidifying and cleaning device 3, since the time information is set such that the content of the hypochlorous acid contained in the hypochlorous acid water stored in humidifier tank 3a is maintained higher than the reference content, the hypochlorous acid of the set concentration can be stably imparted to the air released from the centrifugal crushing unit 3g.

(4) In humidifying and cleaning device 3, a pH adjusting agent for adjusting the pH of the hypochlorous acid water is added to the hypochlorous acid water stored in humidifier tank 3a.

Thus, the hypochlorous acid can be stably imparted from humidifying and cleaning device 3 using the hypochlorous acid water that is easily vaporized by adjusting the pH of the hypochlorous acid water.

### Third Exemplary Embodiment

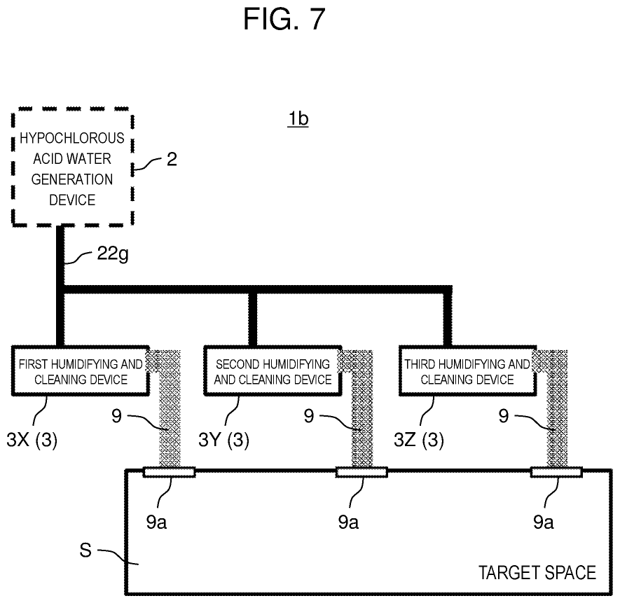

Space cleaning system 1b according to the third exemplary embodiment of the present disclosure will be described with reference to FIG. 7. FIG. 7 is a schematic diagram of space cleaning system 1b according to the third exemplary embodiment of the present disclosure.

A space cleaning system 1b according to the third exemplary embodiment of the present disclosure is different from the space cleaning system of the second exemplary embodiment in that, a plurality of humidifying and cleaning devices 3 is connected to one hypochlorous acid water generation device 2. A basic configuration and a control method of space cleaning system 1b other than this are similar to those of space cleaning system 1a according to the second exemplary embodiment.

In the following, description of the contents already described in the second exemplary embodiment will be omitted as appropriate, and differences from the second exemplary embodiment will be mainly described.

As shown in FIG. 7, space cleaning system 1b includes one hypochlorous acid water generation device 2 and three humidifying and cleaning devices 3. Then, in space cleaning system 1b, the start timings of the micronization operation (a humidification cleaning operation) in the three humidifying and cleaning devices 3 are controlled to be different from each other.

Specifically, space cleaning system 1b includes, as humidifying and cleaning device 3, three devices of first humidifying and cleaning device 3X, second humidifying and cleaning device 3Y, and third humidifying and cleaning device 3Z for sterilizing and deodorizing target space S that is a relatively wide space. Then, each (first humidifying and cleaning device 3X, second humidifying and cleaning device 3Y, and third humidifying and cleaning device 3Z) of the humidifying and cleaning devices 3 is connected to hypochlorous acid water generation device 2 by branched second water supply pipe 22g, and is configured to receive supply of hypochlorous acid water.

Here, each of the humidifying and cleaning devices 3 has the same configuration as humidifying and cleaning device 3 in the second exemplary embodiment, and the humidification control is performed by the same control method. In other words, each of the humidifying and cleaning devices 3 is controlled to execute the first treatment (processing of draining the hypochlorous acid water stored in humidifier tank 3a and supplying new hypochlorous acid water) at preset time intervals (for example, one hour). However, in space cleaning system 1b, the start timing of the micronization operation (the humidification cleaning operation) in each of the humidifying and cleaning devices 3 is controlled to be shifted by a predetermined time (for example, 20 minutes).

In addition, first humidifying and cleaning device 3X corresponds to the “first space cleaning device” of the claims, and second humidifying and cleaning device 3Y corresponds to the “second space cleaning device” of the claims.

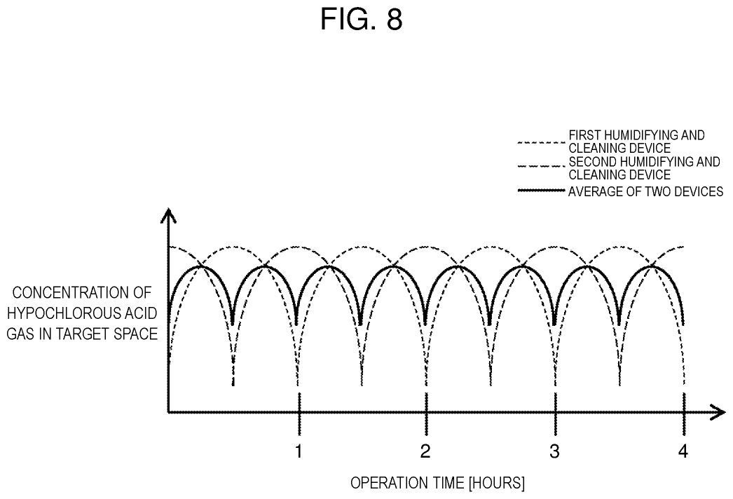

Next, the temporal change in the hypochlorous acid concentration in space cleaning system 1b will be described with reference to FIG. 8. FIG. 8 is a schematic view showing the temporal change in the hypochlorous acid concentration in space cleaning system 1b.

More specifically, FIG. 8 shows the temporal change in the concentration of the hypochlorous acid gas contained in the air in target space S, in a case where the start timings of the humidifying and cleaning are controlled to be shifted by 30 minutes using two humidifying and cleaning devices 3 (for example, first humidifying and cleaning device 3X and second humidifying and cleaning device 3Y). In addition, in FIG. 8, an average concentration of the hypochlorous acid gas of the two devices is indicated by a solid line.

As shown in FIG. 8, in each of first humidifying and cleaning device 3X and second humidifying and cleaning device 3Y, the concentration of the hypochlorous acid gas contained in the air in target space S repeatedly decreases and increases every hour. On the other hand, by shifting the start timings of the humidification cleaning by 30 minutes, a concentration decrease peak of the hypochlorous acid gas of first humidifying and cleaning device 3X and a concentration increase peak of the hypochlorous acid gas of second humidifying and cleaning device 3Y overlap with each other. Therefore, in the average concentration of the hypochlorous acid gas of the two devices, an increase or decrease fluctuation range of the concentration becomes small. In other words, in space cleaning system 1b, the decrease in the hypochlorous acid can be compensated by the two humidifying and cleaning devices 3, and the concentration of the hypochlorous acid contained in the air in target space S can be stabilized.

In addition, the same applies to the three humidifying and cleaning devices 3 including third humidifying and cleaning device 3Z, but as humidifying and cleaning devices 3 that compensate for the decrease in the hypochlorous acid of each other, humidifying and cleaning devices 3 at positions adjacent to each other (positions at close physical distances) are preferable.

As described above, according to space cleaning system 1b according to the third exemplary embodiment, the following effects can be obtained.

(1) In space cleaning system 1b, first humidifying and cleaning device 3X is one of the plurality of humidifying and cleaning devices 3, and the plurality of humidifying and cleaning devices 3 includes first humidifying and cleaning device 3X and second humidifying and cleaning device 3Y different from first humidifying and cleaning device 3X. The hypochlorous acid water generation device 2 is connected to the plurality of humidifying and cleaning devices 3 installed in predetermined target space S so as to be able to supply the hypochlorous acid water. First humidifying and cleaning device 3X and second humidifying and cleaning device 3Y are controlled such that the operation start timings of centrifugal crushing unit 3c after the first treatment are different from each other.

Thus, by the hypochlorous acid released from centrifugal crushing unit 3c of first humidifying and cleaning device 3X and the hypochlorous acid released from centrifugal crushing unit 3c of second humidifying and cleaning device 3Y, the concentration fluctuation range of the hypochlorous acid contained in the air in predetermined target space S can be reduced. In other words, it is possible to provide space cleaning system 1b capable of stabilizing the concentration of the hypochlorous acid contained in the air in predetermined target space S, in a case where the micronization operation of including the hypochlorous acid in the micronized water and releasing the hypochlorous acid is continuously performed.

The present disclosure has been described above based on the exemplary embodiments. It will be understood by those skilled in the art that the exemplary embodiments are merely examples; further, in modifications of the exemplary embodiments, components or processes of the exemplary embodiments are variously combined, and additionally, the modifications fall within the scope of the present disclosure.

In humidifying and cleaning device 3 according to the first exemplary embodiment, hypochlorous acid water concentration sensor 3g detects the concentration of the hypochlorous acid contained in the hypochlorous acid water in humidifier tank 3a at preset time intervals (for example, one minute), but the present disclosure is not limited thereto. For example, the concentration of the hypochlorous acid gas contained in the air flowing through duct 9 (air containing water and the hypochlorous acid) may be detected at preset time intervals (for example, one minute) in duct 9 communicating air delivery port 3e and blow-out port 9a. Then, in a case where the detected concentration of the hypochlorous acid gas is less than or equal to the reference concentration, the first treatment (processing of draining the hypochlorous acid water having a decreased content of the hypochlorous acid in humidifier tank 3a and supplying new hypochlorous acid water having the set concentration) described above may be executed. Even in this case, the effects described above can be obtained.

Further, in humidifying and cleaning device 3 according to the present exemplary embodiment, the humidifying and cleaning is performed using centrifugal crushing unit 3c, but the present disclosure is not limited thereto. For example, a humidification method may be other methods such as an ultrasonic method, a heating method, and a vaporization method.

### INDUSTRIAL APPLICABILITY

The space cleaning device according to the present disclosure and the space cleaning system using the same are useful as a device or a system for sterilizing air in a target space, since the device or the system performs a control capable of stably imparting the hypochlorous acid in a case where the micronization operation of including the hypochlorous acid in the micronized water and releasing the hypochlorous acid is continuously performed.

### REFERENCE MARKS IN THE DRAWINGS

1 space cleaning system
1a space cleaning system
1b space cleaning system
2 hypochlorous acid water generation device
3 humidifying and cleaning device
3a humidifier tank
3b humidifier tank water level sensor
3c centrifugal crushing unit
3c1 pumping pipe
3d air introduction port
3e air delivery port
3f blower
3g hypochlorous acid water concentration sensor
3h drain pipe
3i drain valve
4 hypochlorous acid controller
4a input unit
4b storage unit
4c timer
4d processor
4e output unit
5 humidification controller
5a input unit
5b storage unit
5c timer
5d processor
5e output unit
9 duct
9a blow-out port
10 operation panel
11 temperature and humidity sensor
12a electrolyzer
12b electrode
12c first water pipe
12d chloride ion tank
12e electrolyzer water level sensor
12f first water valve
12g first water supply pipe
12h first water stop valve
12i first pump
22a dilution tank
22b second water pipe
22c dilution tank water level sensor
22d pH adjusting agent tank
22e second water valve
22f second water stop valve
22g second water supply pipe
22h second pump

1. A space cleaning device comprising:
a humidifying and cleaning unit configured to generate hypochlorous acid water micronized by a micronization operation of micronizing the hypochlorous acid water stored in a water storage unit, and causes air flowing inside of the humidifying and cleaning unit to contain the hypochlorous acid water micronized by the micronization operation and release the hypochlorous acid water; and
a controller configured to control the micronization operation,
wherein the controller is configured to perform a first treatment of draining the hypochlorous acid water stored in the water storage unit and supplying new hypochlorous acid water during the micronization operation, based on time information specified in advance, that is, the time information from when the micronization operation is started until a content of the hypochlorous acid contained in the hypochlorous acid water stored in the water storage unit becomes less than or equal to a reference content.

2. The space cleaning device according to claim 1, wherein the humidifying and cleaning unit generates the micronized the hypochlorous acid water by the micronization operation of centrifugally crushing and micronizing the hypochlorous acid water pumped from the water storage unit by rotation of a pumping pipe.

3. The space cleaning device according to claim 1, wherein the time information is specified in advance for each concentration of the hypochlorous acid water stored in the water storage unit.

4. The space cleaning device according to claim 1, wherein a pH adjusting agent for adjusting a pH of the hypochlorous acid water is added to the hypochlorous acid water stored in the water storage unit.

5. A space cleaning system comprising:
the space cleaning device according to claim 1; and
a hypochlorous acid water generation device configured to generate hypochlorous acid water by electrolyzing a chloride aqueous solution,
wherein the hypochlorous acid water generation device supplies the hypochlorous acid water to the water storage unit in the first treatment.

6. The space cleaning system according to claim 5, wherein
the space cleaning device is one of a plurality of space cleaning devices, and the plurality of the space cleaning devices include a first space cleaning device that is the space cleaning device and a second space cleaning device different from the first space cleaning device;
the hypochlorous acid water generation device is connected to the plurality of the space cleaning devices installed in a predetermined target space, being able to supply the hypochlorous acid water; and
the first space cleaning device and the second space cleaning device are controlled, operation start timings of the humidifying and cleaning unit after the first treatment being different from each other.

7. A space cleaning device comprising:
a humidifying and cleaning unit configured to generate hypochlorous acid water micronized by a micronization operation of centrifugally crushing and micronizing the hypochlorous acid water pumped from a water storage unit by rotation of a pumping pipe, cause air flowing inside to contain and release the hypochlorous acid water micronized by the micronization operation; and
a controller configured to control the micronization operation,
wherein the controller is configured to cause the water storage unit to execute a wastewater treatment of the hypochlorous acid water stored in the water storage unit during the micronization operation, based on time information specified in advance, that is, time information until a content of the hypochlorous acid contained in the hypochlorous acid water stored in the water storage unit becomes less than or equal to a reference content.

8. The space cleaning device according to claim 7, wherein the time information is specified in advance for each concentration of the hypochlorous acid water stored in the water storage unit.

9. The space cleaning device according to claim 7, wherein a pH adjusting agent for adjusting a pH of the hypochlorous acid water is added to the hypochlorous acid water stored in the water storage unit.
